(19) United States
(12) Patent Application Publication
(10) Pub. No.: US 2024/0390046 A1
(43) Pub. Date: Nov. 28, 2024

(54) IMPLANTS, SYSTEMS AND METHODS OF USING THE SAME

(71) Applicant: David Brian KORNREICH, Mequon, WI (US)

(72) Inventor: David Brian KORNREICH, Mequon, WI (US)

(21) Appl. No.: 18/795,137

(22) Filed: Aug. 5, 2024

Related U.S. Application Data
(63) Continuation of application No. PCT/US2023/062081, filed on Feb. 6, 2023.
(60) Provisional application No. 63/307,090, filed on Feb. 5, 2022.

(51) Int. Cl. A61B 17/80; A61B 17/56; A61B 17/68

(52) U.S. Cl. CPC A61B 17/8061; A61B 17/683; A61B 2017/564

(57) ABSTRACT

Implants, devices, systems and methods for replacing bone fixation, such as scapula fixation. A bone plate system includes a plate and at least one fastener for insertion through the plate. The plate with a body portion, a base portion positioned inferior to and spaced apart from the body portion, and a connecting member coupled to the body portion on a first end and the base portion on a second end. Methods for implanting the implant are also disclosed.

### CROSS-REFERENCE TO RELATED APPLICATION

This application is a continuation of PCT/US2023/062081 filed on Feb. 6, 2023, which claims priority benefit under 35 U.S.C. § 119 (e) of U.S. provisional application No. 63/307,090 filed Feb. 5, 2022, which are incorporated herein by reference in their entireties.

### FIELD OF THE INVENTION

The present invention relates generally to general surgery, orthopedic systems and implants used for bone fixation, such as scapula fixation. More specifically, but not exclusively, the present invention relates to plating system and implants for acromial fractures, as well as methods for using the same.

### BACKGROUND OF THE INVENTION

The acromion is a portion of the scapula which acts as a bony area of origination for several muscles in the shoulder.

Within the past decade a new shoulder arthroplasty design, i.e., a reverse total shoulder arthroplasty, has been used to solve the previously difficult problem of dysfunction in shoulders with a deficient rotator cuff. This technique increased burden on the deltoid in order to power the shoulder and along with that the acromion process of the scapula from which the deltoid originates.

Hence, an increasing number of acromial fractures have been seen. Fixation of acromial fractures in this typically osteoporotic patient population is difficult and current plate designs do not adequately address the unique characteristics and biomechanical stresses of this anatomic area.

Thus, what is needed is an improved plating system that provides a stable and more robust fixation of these fractures which will increase the chance of bony union and improved patient function.

### SUMMARY OF THE INVENTION

Aspects of the present disclosure provide glenoid implants for anatomic shoulder arthroplasties. The present invention also provides for methods for using the glenoid implants.

In one aspect, provided herein is a bone plate system, including a plate and at least one fastener for insertion through the plate. The plate including a body portion, a base portion positioned inferior to and spaced apart from the body portion, and a connecting member coupled to the body portion on a first end and the base portion on a second end.

In another aspect, provided herein is a plate. The plate including a body portion, a base portion positioned inferior to and spaced apart from the body portion, and a connecting member coupled to the body portion on a first end and the base portion on a second end.

In yet another aspect, provided herein is a surgical method of using a plate. The method including surgically exposing a desired bone and making a slit in a deltoid. The method also includes inserting the plate with a base portion inserted through the slit in the deltoid and under an acromion. Further, the method includes inserting at least one fastener to secure the plate.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a first perspective view of a bone plate system, in accordance with an aspect of the present disclosure;

FIG. 2 is a second perspective view of the bone plate system of FIG. 1, in accordance with an aspect of the present disclosure;

FIG. 3 is an exploded, perspective view of the bone plate system of FIG. 1, in accordance with an aspect of the present disclosure;

FIG. 4 is a first perspective view of a plate from the bone plate system of FIG. 1, in accordance with an aspect of the present disclosure;

FIG. 5 is a second perspective view of the plate of FIG. 4, in accordance with an aspect of the present disclosure;

FIG. 6 is a first side view of the plate of FIG. 4, in accordance with an aspect of the present disclosure;

FIG. 7 is a second side view of the plate of FIG. 4, in accordance with an aspect of the present disclosure;

FIG. 8 is a first end view of the plate of FIG. 4, in accordance with an aspect of the present disclosure;

FIG. 9 is a second end view of the plate of FIG. 4, in accordance with an aspect of the present disclosure;

FIG. 10 is a superior view of the plate of FIG. 4, in accordance with an aspect of the present disclosure;

FIG. 11 is an inferior view of the plate of FIG. 4, in accordance with the present disclosure;

FIG. 12 is a first perspective view of another plate, in accordance with an aspect of the present disclosure;

FIG. 13 is a second perspective view of the plate of FIG. 12, in accordance with an aspect of the present disclosure;

FIG. 14 is a first perspective view of yet another plate, in accordance with an aspect of the present disclosure;

FIG. 15 is a second perspective view of the plate FIG. 14, in accordance with an aspect of the present disclosure;

FIG. 16 is a posterior view of another plate placed on a scapula with a supra-acromial, in accordance with an aspect of the present disclosure;

FIG. 17 is an inferio-posterior view of the plate of FIG. 16 on a scapula, in accordance with an aspect of the present disclosure; and

FIG. 18 is a lateral view of the plate of FIG. 16 on a scapula, in accordance with an aspect of the present disclosure.

### DETAILED DESCRIPTION FOR CARRYING OUT THE INVENTION

Generally stated, disclosed herein are plating systems and implants for fixing acromial fractures. Further, surgical methods for using systems and implants are also discussed.

In this detailed description and the following claims, the words proximal, distal, anterior, posterior, medial, lateral, superior and inferior are defined by their standard usage for indicating a particular part of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

Similarly, positions or directions may be used herein with reference to anatomical structures or surfaces. For example, as the current implants, devices, systems and methods are described herein with reference to use with the bones of the shoulder, the bones of the shoulder and upper arm may be used to describe the surfaces, positions, directions or orientations of the implants, devices, systems and methods. Further, the implants, devices, systems and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, systems and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, systems and methods, and the aspects, components, features and the like thereof, described herein with respect to the right shoulder may be mirrored so that they likewise function with the left shoulder and vice versa. Further, the implants, devices, systems and methods, and the aspects, components, features and the like thereof, disclosed herein are described with respect to the shoulder for brevity purposes, but it should be understood that the implants, devices, systems and methods may be used with other bones of the body having similar structures, for example the lower extremity, and more specifically, with the bones of the ankle, foot, and leg.

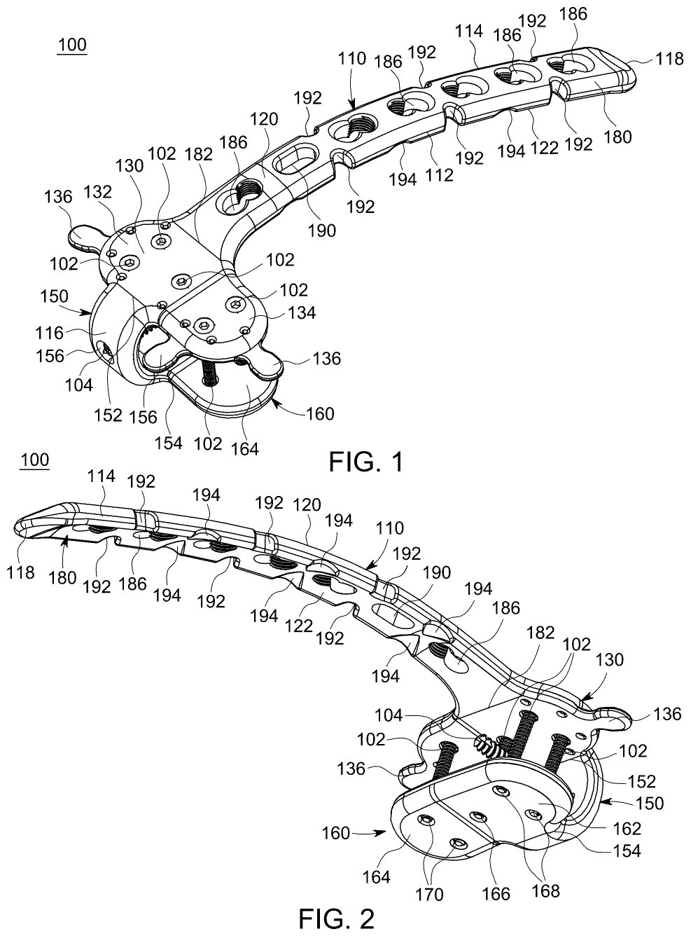

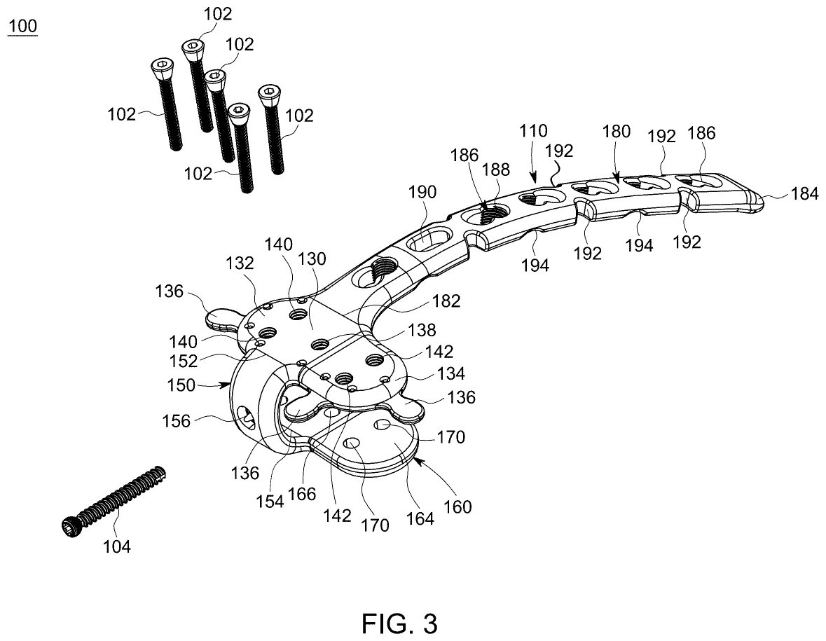

Referring to the drawings, wherein like reference numerals are used to indicate like or analogous components throughout the several views, and with particular reference to FIGS. 1-3, there is illustrated an embodiment of a bone plate system or acromial plating system 100. The system 100 includes an implant or bone plate 110, at least one fastener 102 for insertion through the bone plate 110, and a locking screw 104 for insertion through the bone plate 110. The at least one fastener 102 may be, for example, five fasteners 102. The fasteners 102 may be, for example, screws, cortical screws, bone screws, cables, wires, or the like. The screws may be, for example, threaded along a portion of the length or the entire length. The locking screw 104 may, for example, include threads along at least a portion of its shaft. In addition, the locking screw 104 may include threads on at least a portion of the head.

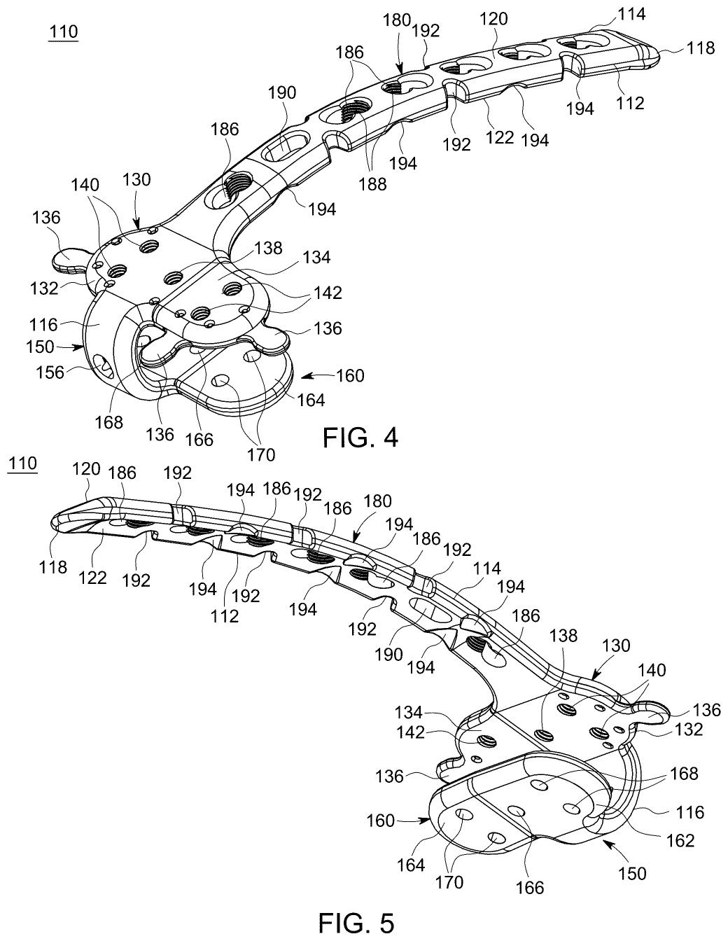

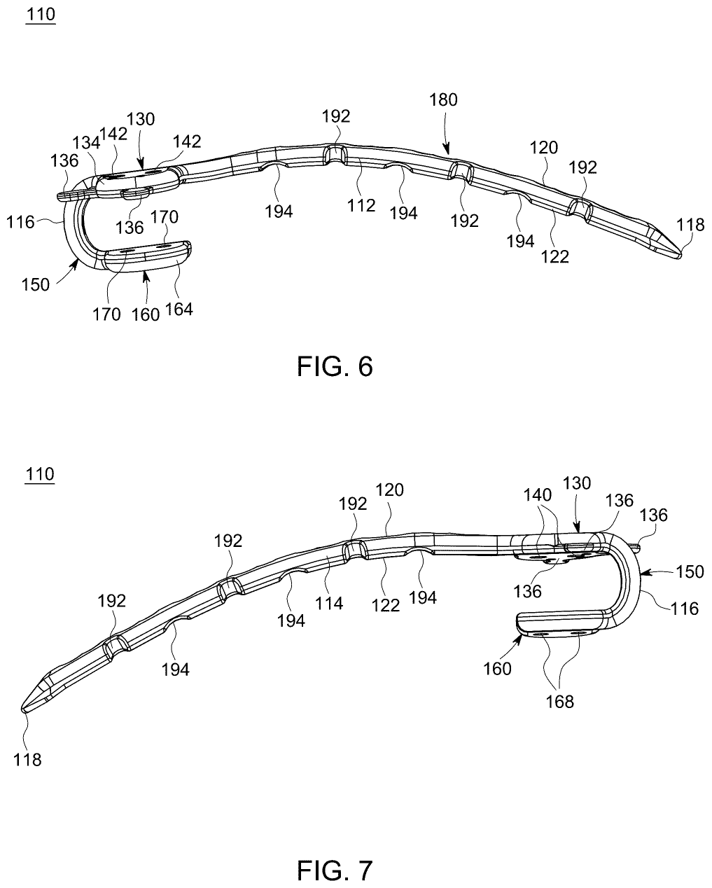

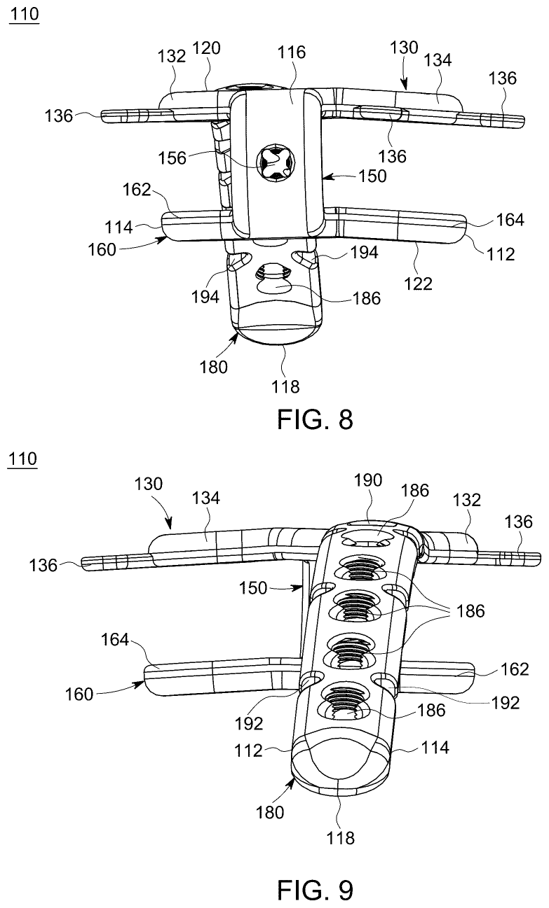

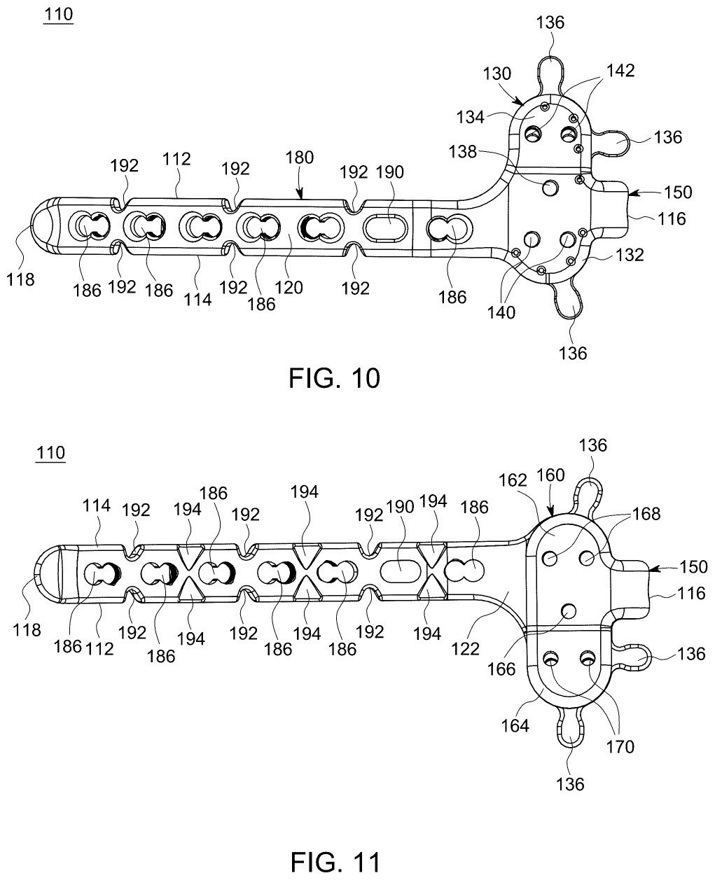

With continued reference to FIGS. 1-3 and reference to FIGS. 4-11, the plate 110 is shown. The plate 110 includes a first or anterior side 112 opposite a second or posterior side 114, a first or lateral end 116 opposite a second or medial end 118, and a top or superior surface 120 opposite a bottom or inferior surface 122. The plate 110 also includes a body portion 130, a connecting member 150, a base portion 160, and an extension portion 180. The body portion 130 is positioned near the first end 116 of the plate 110. The base portion 160 is positioned inferior to and spaced apart from the body portion 130. The connecting member 150 couples the body portion 130 and the base portion 160 and extends from the body portion 130 to the base portion 160. The connecting member 150 is coupled to a first end of the body portion 130. The extension portion 180 extends away from the body portion 130 on a second end.

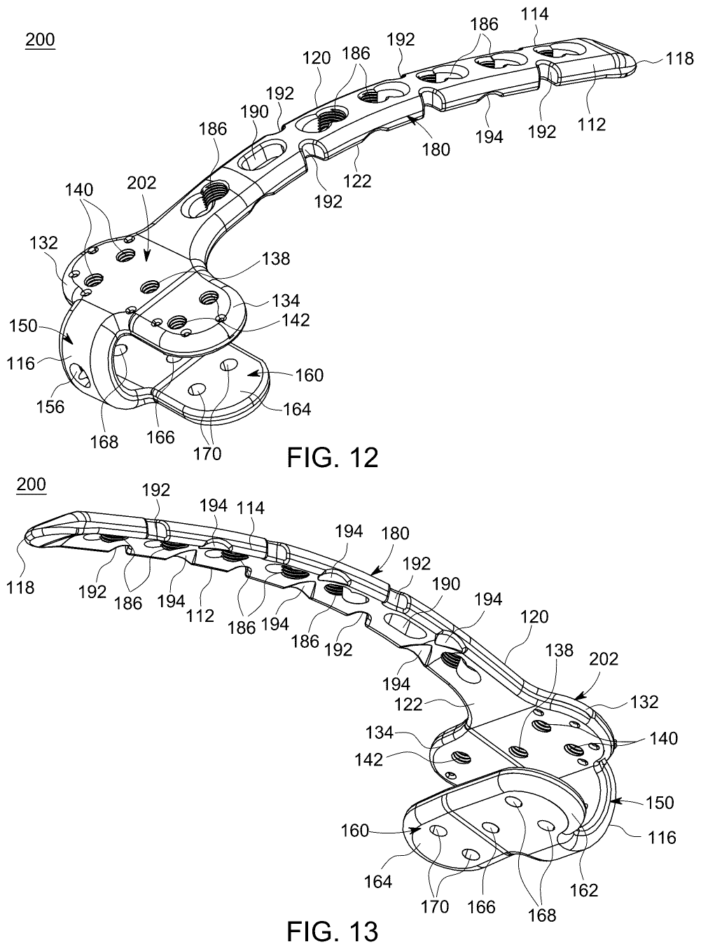

With continued reference to FIGS. 1-11, the body portion 130 of the plate 110 includes a first portion 132 at a first side and a second portion 134 at a second side. The plate 110 may be, for example, oval or oblong. The plate 110 may have, for example, rounded ends and flat or planar long sides. The first portion 132 may extend out from the second side 114 of the plate 110 beyond the connecting member 150 and the extension portion 180. The first portion 132 may extend away from the extension portion 180 a first length. The second portion 134 may extend out from the first side 112 of the plate 110 beyond the connecting member 150 and the extension portion 180. The second portion 134 may extend away from the extension portion a second length. The first length of the first portion 132 may be, for example, smaller than the second length of the second portion 134. In some embodiments, the plate 110 may also include at least one tab 136 extending away from an exterior surface of the body portion 130, as shown in FIGS. 1-11. Alternative embodiments, may not include the tabs 136, such as plate 200, as shown in FIGS. 12-13 and described in greater detail below. With continued reference to FIGS. 1-11, the at least one tab 136 may be, for example, three tabs 136. The plate 110 may include a first tab 136 extending away from the first portion 312 of the body portion 130. The plate 110 may also include a second tab 136 and third tab 136 extending away from the second portion 134. The second tab 136 may extend away from an anterior side 112 of the second portion 134 and the third tab 136 may extend away from a lateral end 116 of the second portion 134. The body portion 130 of the plate 110 may also include a plurality of openings 138, 140, 142 spaced across the body portion 130. The plurality of the openings 138, 140, 142 may include a first opening 138 positioned intermediate the first portion 132 and the second portion 134 of the body portion 130. The first opening 138 may be, for example, positioned at a midpoint between a first side 112 and a second side 114 of the plate 110. The first opening 138 extends through the body portion 130 from a top surface 120 to a bottom surface 122. The plurality of the openings 138, 140, 142 may also include at least one second opening 140. The at least one second opening 140 may be, for example, two second openings 140 positioned in the first portion 132 of the body portion 130. The two second openings 140 may be, for example, spaced apart from each other along a length between the first end 116 and the second end 118. The second openings 140 extend through the body portion 130 from a top surface 120 to a bottom surface 122. The plurality of the openings 138, 140, 142 may also include at least one third opening 142. The at least one third opening 142 may be, for example, two third openings 142 positioned in the second portion 134 of the body portion 130. The two third openings 142 may be, for example, spaced apart from each other along a length between the first end 116 and the second end 118. The third openings 142 extend through the body portion 130 from the top surface 120 to the bottom surface 122. The plurality of the openings 138, 140, 142 are configured or sized and shaped to receive a fastener 102.

The connecting member 150 is also shown in FIGS. 1-11. The connecting member 150 includes a first end 152 and a second end 154. The first end 152 is coupled to and extends away from the lateral end 116 of the body portion 130. The second end 154 is coupled to and extends away from the lateral end 116 of the base portion 160. As shown in FIGS. 1-11, the connecting member 150 may be, for example, directly coupled to the body portion 130 on one end and the base portion 160 on the other end. However, in alternative embodiments, the connecting member 150 may be, for example, removably coupled to the body portion 130 and base portion 160. The connecting member 150 may be, for example, coupled to the body portion 130 and base portion 160 during insertion into the patient and then once the body portion 130 and base portion 160 are fixed to the patient's bone, the connecting member 150 may be removed from the patient. The connecting member 150 may be coupled to the body portion 130 and base portion 160 by, for example, tongue and groove, dovetail, protrusion and slot, or another known mechanical attachment means allowing for securement during insertion and removal after insertion is complete.

As shown in FIGS. 1-11, in some embodiments the connecting member 150 may extend from the body portion 130 offset from a center of the body portion 130. Although alternative positioning of the connecting member 150 to the body portion 130 are also contemplated, for example, the connecting member 150 may extend from a central part of the body portion 130 or be offset either anteriorly or posteriorly from a midpoint of the body portion 130. The connecting member 150 extends from at least the first portion 132 of the body portion 130. The connecting member 150 may also extend from the base portion 160 offset from a center of the base portion 160 in some embodiments. In alternative embodiments, it is also contemplated that the connecting member 150 may extend from, for example, a central part of the base portion 160 or be offset either anteriorly or posteriorly from a midpoint of the base portion 160. The connecting member 150 further extends from at least the first portion 162 of the base portion 160. The connecting member 150 may be, for example, curved or arced as the connecting member 150 extends between the body portion 130 and the base portion 150. The connecting member 150 may have, for example, a C-shape. The connecting member 150 may also have an opening 156 extending through the connecting member 150 from an exterior surface to an interior surface. The opening 156 may be positioned, for example, at a midpoint of the connecting member 150 between the first end 152 and the second end 154. The opening 156 may be, for example, configured or sized and shaped to receive a locking screw 104.

As shown in FIGS. 1-11, the base portion 160 of the plate 110 includes a first portion 162 at a first side and a second portion 164 at a second side. The plate 110 may be, for example, oval or oblong. The plate 110 may have, for example, rounded ends and flat or planar long sides. The first portion 162 may extend out from the second side 114 of the plate 110 beyond the connecting member 150 and the extension portion 180. The first portion 162 may extend away from the extension portion 180 a first length. The second portion 164 may extend out from the first side 112 of the plate 110 beyond the connecting member 150 and the extension portion 180. The second portion 164 may extend away from the extension portion 180 a second length. The first length of the first portion 162 may be, for example, smaller than the second length of the second portion 164. The body portion 130 of the plate 110 may also include a plurality of openings 166, 168, 170 spaced across the base portion 160. The plurality of the openings 166, 168, 170 may include a first opening 166 positioned intermediate the first portion 162 and the second portion 164 of the base portion 160. The first opening 166 may be, for example, positioned at a midpoint between a first side 112 and a second side 114 of the plate 110. The first opening 166 extends through the base portion 160 from a top surface 120 to a bottom surface 122. The plurality of the openings 166, 168, 170 may also include at least one second opening 168. The at least one second opening 168 may be, for example, two second openings 168 positioned in the first portion 162 of the base portion 160. The two second openings 168 may be, for example, spaced apart from each other along a length between the first end 116 and the second end 118. The second openings 168 extend through the base portion 160 from a top surface 120 to a bottom surface 122. The plurality of the openings 166, 168, 170 may also include at least one third opening 170. The at least one third opening 170 may be, for example, two third openings 170 positioned in the second portion 164 of the base portion 160. The two third openings 170 may be, for example, spaced apart from each other along a length between the first end 116 and the second end 118. The third openings 170 extend through the base portion 160 from the top surface 120 to the bottom surface 122. The plurality of the openings 166, 168, 170 are configured or sized and shaped to receive a fastener 102.

As shown in FIGS. 1-11, the body portion 130 may be positioned spaced apart from and directly over the base portion 160. The body portion 130 may be spaced apart from the base portion 160 based on, for example, the height of the connecting member 150. The body portion 130 may be positioned to align the openings 138, 140, 142 with the openings 166, 168, 170 of the base portion 160 to allow the fasteners 102 to extend through the body portion 130 and into the base portion 160. Although the body portion 130 and base portion 160 are shown as mirror image of each other in the illustrated embodiments, it is also contemplated that the base portion 160 may have an alternative shape or smaller size than the body portion 130. For example, the alternative shape may be a dog bone shape or other shape providing only enough surface to include the openings 166, 168, 170 for coupling the base portion 160 with the body portion 130.

With continued reference to FIGS. 1-11, the extension portion 180 is shown and includes a first end 182 and a second end 184. The first end 182 of the extension portion 180 is coupled to and extends away from the body portion 130. The extension portion 180 may, for example, curve or arc along the length of the extension portion 180 between the first end 182 and the second end 184. The extension portion 180 may be, for example, configured or sized and shaped to correspond to the shape of the superior surface of a patient's scapula. The extension portion 180 couples to the body portion 130 offset to the second side 114 of the plate 110. The extension portion 180 couples at least the first portion 132 of the body portion 130. The extension portion 180 includes at least one enlarged bone screw hole 186 positioned along the length of the extension portion 180. The screw holes 186 may include a threaded portion 188 along at least a portion of the hole 186. The threaded portion 188 may be positioned, for example, in a medial or lateral portion of each screw hole 186. The at least one enlarged bone screw holes 186 may be any number of screw holes 186 based upon the length of the extension portion 180, for example, one to six holes 186 or more. The extension portion 180 may also include at least one slot 190 along the length of the extension portion 180 between the first end 182 and the second end 184. The at least one slot 190 may have, for example, an oval or oblong shape. The at least one slot 190 may be, for example, one slot 190. The slot 190 may be positioned along the length of the extension portion 180 aligned with the screw holes 186. The extension portion 180 may also include at least one relief 192 positioned along the length of the extension portion 180 between the first end 182 and the second end 184. Each relief 192 of the at least one relief 192 extends into one of the first side 112 and the second side 114 of the extension portion 180. As shown, the reliefs 192 may be positioned in pairs with a first relief 192 extending into the first side 112 of the extension portion 180 and a second relief 192 extending into the second side 114 of the extension portion 180. The first relief 192 may be, for example, positioned directly opposite the second relief 192. The extension portion 180 may include multiple pairs of reliefs 192 along the length between the first end 182 and the second end 184. The extension portion 180 may further include at least one groove 194 positioned along the length of the extension portion 180 between the first end 182 and the second end 184. Each groove 194 of the at least one groove 194 extends into the extension portion 180 from the inferior surface. As shown, the grooves 194 may be positioned in pairs with a first groove 194 extending into the inferior surface near the first side 112 of the extension portion 180 and a second groove 194 extending into the inferior surface near the second side 114 of the extension portion 180. The first groove 194 may be, for example, positioned directly opposite the second groove 194. The extension portion 180 may include multiple pairs of grooves 194 along the length between the first end 182 and the second end 184. The pairs of reliefs 192 may alternate with the pairs of grooves 194 along the length of the extension portion 180. The reliefs 192 and grooves 194 may allow for curving or bending of the extension portion 180 of the plate 110 during surgery.

Referring now to FIGS. 12-13, another plate 200 is shown. The plate 200 includes a body portion 202, a connecting member 150, a base portion 160, and an extension portion 180. The connecting member 150, the base portion 160, and the extension portion 180 are as described above with reference to plate 110 and which will not be described again here for brevity's sake. The body portion 202 is similar to the body portion 130, however, the body portion 202 lacks the tabs 136, as shown in FIGS. 12-13. The remaining portions of the body portion 202 are as described above with reference to body portion 130 and will also not be disclosed again here for brevity's sake.

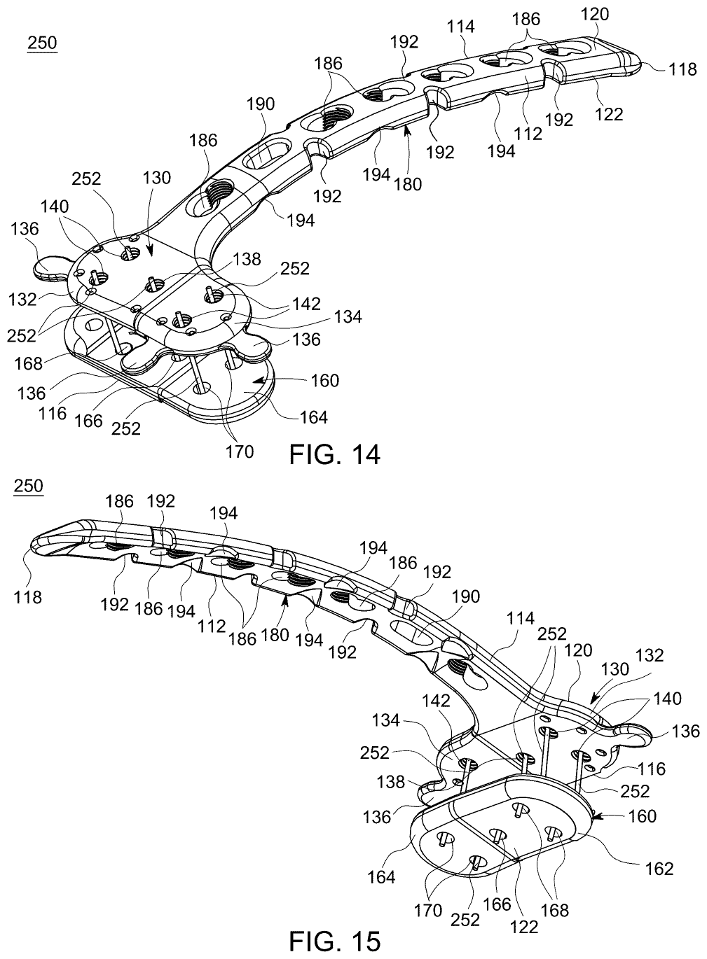

Referring now to FIGS. 14-15, another plate 250 is shown. The plate 250 includes a body portion 130, a base portion 160, an extension portion 180, and at least one coupling member 252 securing the body portion 130 and the base portion 160. The body portion 130, the base portion 160, and the extension portion 180 are as described above with reference to plate 110 and which will not be described again here for brevity's sake. The at least one coupling member 252 may be, for example, five coupling members 160 received within the openings 138, 140, 142 of the body portion 130 and the openings 166, 168, 170 of the base portion 160. The at least one coupling member 252 may be, for example, wires, screws, cables, posts, pegs, or the like. The coupling members 252 may be, for example, secured to each of the body portion 130 and base portion 160 to couple the body portion 130 to the base portion 160.

A method of using the bone plate system 100 includes obtaining the bone plate system 100. Creating an incision in the patient over a surgical site. Preparing a patient's shoulder bones for the surgical procedure. Inserting the plate 110 into the patient and sliding the base portion 160 of the plate 110 through a slit in the deltoid. More specifically, the first side 112 or second portion 164 of the base portion 160 may be positioned to be inserted through the slit in the deltoid. The second portion 164 of the base portion 160 may then be inserted through the slit in the deltoid and the plate 110 may be rotated, for example, counterclockwise approximately 90° to insert the rest of the base portion 160 through the slit in the deltoid and under the lateral aspect of the acromion. The insertion of the base portion 160 may be performed, for example, from the lateral side of the patient. Next, the base portion 160 is positioned under the acromion with the connecting member 150 wrapping around the lateral aspect of the acromion and positioning the remaining portion of the plate 110 on a superior surface of the scapula. Then, fasteners 102 and/or locking screws 104 may be inserted through the plate 110 into a portion of bone. For embodiments with tabs 136, the tabs 136 may be activated to assist with securing the plate 110 to the patient's bone. The tabs 136 may be used before or after inserting the fasteners 102 and/or locking screws 104. Next, additional fasteners or bones screws may be inserted through the extension portion 180 of the plate 110 to secure the extension portion 180 along the scapula. Once all the fasteners and/or screws are inserted into the patient's bones, the surgical procedure may be completed and the patient's incision closed.

The plates 110, 200, 250 are made of sufficient material to attach to the medial scapula with a portion coupling to a subacromial portion. The plates 110, 200, 250 include a portion that lies inferior to the acromion. The acromion is a wafer of often osteoporotic bone. The plates 110, 200, 250 include a portion that supports the acromial bone with a subacromial material which decreases the screw pull out mechanism of failure.

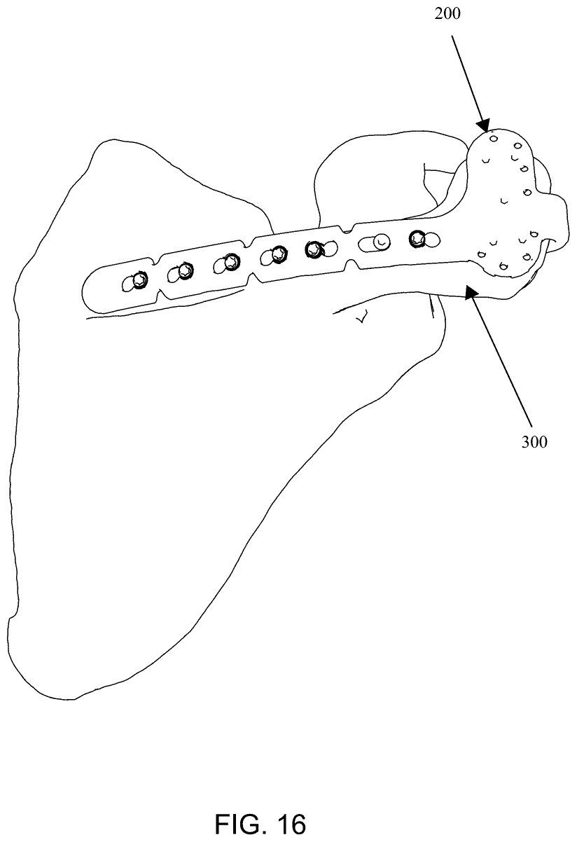

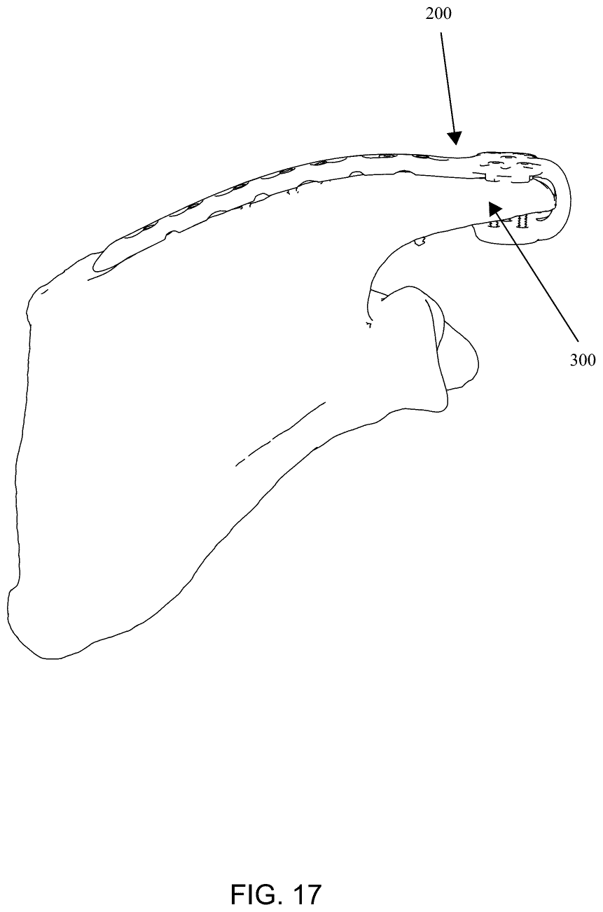

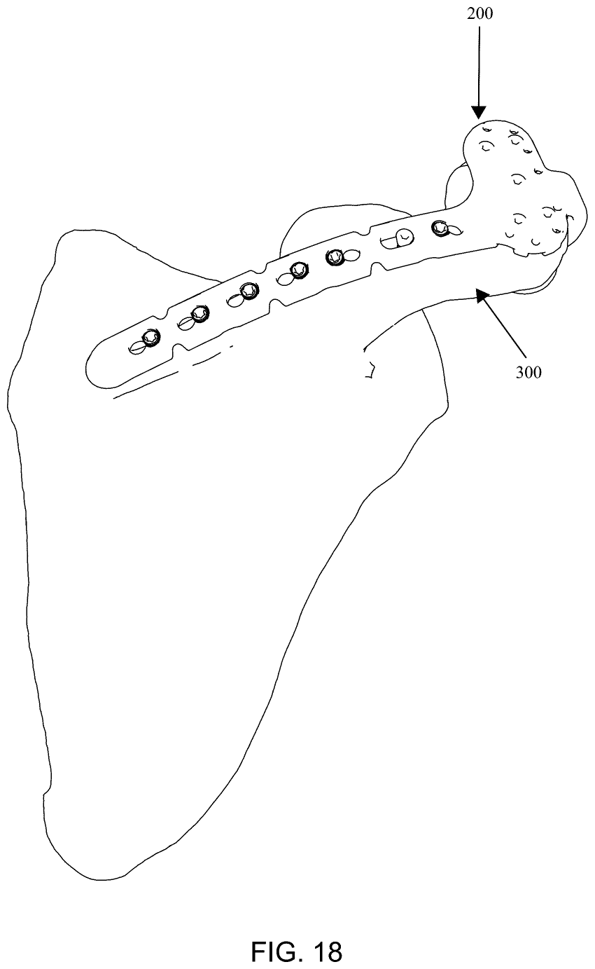

Referring now to FIGS. 16-18, another plate, such as plate 200, is shown. The plate sits both above and below the acromion 300. Specifically, the body portion 130, 202 sit above the acromion 300 and the base portion 160 sit below the acromion 300. The fasteners 102 extend through the body portion 130, 202, the acromion 300, and the base portion 160 to secure the plate 110, 200, 250.

As may be recognized by those of ordinary skill in the art based on the teachings herein, numerous changes and modifications may be made to the above-described and other embodiments of the present disclosure without departing from the scope of the disclosure. The components of the implants, devices, and/or systems as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the implants, devices, and/or systems may include more or fewer components or features than the embodiments as described and illustrated herein. For example, the components and features of implants 110, 200, 250 may be used interchangeably and in alternative combinations as would be modified or altered by one of skill in the art. Further, the steps of the surgical methods associated with the implants 110, 200, 250 may be used interchangeably and in alternative combinations as would be modified or altered by one of skill in the art. Accordingly, this detailed description of the currently-preferred embodiments is to be taken in an illustrative, as opposed to limiting of the disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. A bone plate system, comprising:
a plate, comprising:
a body portion; and
a base portion positioned inferior to and spaced apart from the body portion; and
at least one fastener for insertion through the plate.

2. The system of claim 1, wherein the plate further comprises:
a connecting member coupled to the body portion on a first end and the base portion on a second end; and

3. The system of claim 2, wherein the plate further comprises:
an extension portion coupled to and extending from the body portion.

4. The system of claim 3, wherein the body portion comprises:
a first portion positioned near a first side of the plate; and
a second portion positioned near a second side of the plate.

5. The system of claim 4, wherein the body portion further comprises:
at least one opening for receiving the at least one fastener.

6. The system of claim 5, wherein the at least one opening comprises:
a first opening extending through the body portion near a midpoint of the body portion;
at least one second opening extending through the first portion of the body portion; and
at least one third opening extending through the second portion of the body portion.

7. The system of claim 6, wherein the body portion further comprises:
at least one tab extending away from the first portion of the body portion; and
at least one tab extending away from the second portion of the body portion.

8. The system of claim 5, wherein connecting member further comprising:
an opening extending through the connecting member from an exterior surface to an interior surface.

9. The system of claim 8, wherein the base portion comprises:
a first portion positioned near a first side of the plate;
a second portion positioned near a second side of the plate; and
at least one opening for receiving the at least one fastener.

10. The system of claim 9, wherein the at least one opening comprises:
a first opening extending through the base portion near a midpoint of the base portion;
at least one second opening extending through the first portion of the base portion; and
at least one third opening extending through the second portion of the base portion.

11. The system of claim 2, wherein the body portion is a mirror image of the base portion.

12. The system of claim 3, wherein the extension portion comprises:
at least one screw hole positioned along the length of the extension portion between a first end and a second end; and
at least one slot positioned along the length of the extension portion between the first end and the second end, wherein the at least one screw hole is offset from the at least one slot.

13. The system of claim 3, wherein the extension portion comprises:
at least one relief extending into the extension portion from at least one of a first side and a second side; and
at least one groove extending into an inferior surface of the extension portion.

14. The system of claim 2, wherein the connecting member is removable.

15. A plate, comprising:
a body portion; and
a base portion positioned inferior to and spaced apart from the body portion, wherein the body portion is coupled to the base portion by at least one fastener.

16. The plate of claim 15, wherein the plate further comprises:
a connecting member coupled to the body portion on a first end and the base portion on a second end, wherein connecting member further comprising:
an opening extending through the connecting member from an exterior surface to an interior surface; and
an opening extending through the connecting member from the exterior surface to the interior surface;
wherein the connecting member is removable;
an extension portion coupled to and extending from the body portion, wherein the body portion is a mirror image of the base portion;
wherein a midpoint of the connecting member is offset from a midpoint of the body portion and a midpoint of the base portion;
wherein a midpoint of the extension portion is offset from a midpoint of the body portion as the extension portion extends from the body portion.

17. The plate of claim 16, wherein the body portion comprises:
a first portion positioned near a first side of the plate;
a second portion positioned near a second side of the plate;
at least one opening extending through the body portion, wherein the at least one opening comprises:
a first opening extending through the body portion near a midpoint of the body portion;
at least one second opening extending through the first portion of the body portion; and
at least one third opening extending through the second portion of the body portion.
at least one tab extending away from the first portion of the body portion; and
at least one tab extending away from the second portion of the body portion.

18. The plate of claim 16, wherein the base portion comprises:
a first portion positioned near a first side of the plate;
a second portion positioned near a second side of the plate; and
at least one opening extending through the base portion, wherein the at least one opening comprises:
a first opening extending through the base portion near a midpoint of the base portion;
at least one second opening extending through the first portion of the base portion; and
at least one third opening extending through the second portion of the base portion.

19. The plate of claim 17, wherein the extension portion comprises:
at least one screw hole positioned along the length of the extension portion between a first end and a second end;
at least one slot positioned along the length of the extension portion between the first end and the second end, wherein the at least one screw hole is offset from the at least one slot;
at least one relief extending into the extension portion from at least one of a first side and a second side; and
at least one groove extending into an inferior surface of the extension portion.

20. A method of using a plate, comprising:
surgically exposing a desired bone;
making a slit in a deltoid;
inserting the plate with a base portion inserted through the slit in the deltoid and under an acromion; and
inserting at least one fastener to secure the plate.
